(19) United States
(12) Patent Application Publication
(10) Pub. No.: US 2014/0378954 A1
(43) Pub. Date: Dec. 25, 2014

(54) DEVICES HAVING A LASER FOR CLOSING OPEN WOUNDS AND FOR PROCESSING TISSUE OF A HUMAN OR ANIMAL BODY

(71) Applicant: Technische Universität Ilmenau, Ilmenau (DE)

(72) Inventors: Andreas Schober, Erfurt (DE); Michael Gebinoga, Ilmenau (DE); Uta Fernekorn, Erfurt (DE)

(21) Appl. No.: 14/369,004

(22) PCT Filed: Dec. 15, 2012
(86) PCT No.: PCT/EP2012/075678

§ 371 Date: Jun. 26, 2014

(30) Foreign Application Priority Data
Dec. 30, 2011 (DE) 10 2011 057 184.1

(51) Int. Cl. A61B 17/00; A61N 5/06; A61L 24/00; A61B 18/22

(52) U.S. Cl. CPC A61B 17/00491; A61B 18/22; A61N 5/0613; A61L 24/0005; A61B 2017/00508; A61B 2017/00517; A61B 2017/00522; A61B 2017/0065; A61N 2005/0602; A61N 2005/0659; A61N 2005/067
USPC 606/2

(57) ABSTRACT

The present invention relates to a device for closing an open, bleeding wound of an animal or human body. The invention further relates to a device for processing tissue of a human or animal body, for example, a device for producing a support in a vessel of the body. The device for closing a bleeding wound firstly comprises a laser for irradiating the blood in the wound with infrared laser radiation. According to the invention, the laser radiation of the laser can be adjusted such that two- or multiphoton absorption occurs in irradiated regions of the blood, whereby the blood in the irradiated regions polymerises into a solidified biopolymer and closes the wound.

### FIELD OF THE INVENTION

The present invention relates first of all to a device for closing an open bleeding wound of an animal or human body. The invention also relates to a device for processing tissue of a human or animal body, for example, a device for creating a support in a vessel of the body. The invention may also be used, for example, for connecting pieces of tissue or tissue flaps in the manner of attaching or adhering, whereby such connections may be made not only to pieces of tissue in the body, but outside the body as well, for example to removed tissue or artificially produced tissue.

### BACKGROUND OF THE INVENTION

DE 101 02 477 A1 shows a device for laser welding two vessels. This device may be used, for example, to direct laser light at a wave length of 808 nm at the connection site, at which biological solder is situated. The tissue of the vessels to be connected is melted.

EP 1 885 270 B1 shows a device for welding and cutting tissue, which includes two heating elements. The tissue is melted as it is being welded.

WO 2010/033765 A1 and WO 2006/057784 A2 show other methods for laser welding tissue in which the tissue is melted.

U.S. Pat. No. 7,077,839 B2 shows a method for welding tissue using a protein solder which is activatable by a laser. In this method as well, the result is a denaturing of the tissue and of the solder used.

A method for gluing tissue is known from U.S. Pat. No. 6,939,364 B1 in which an adhesive having collagen is used. The collagen is exposed to radiation, for example, laser radiation, which results in the denaturing of the collagen.

U.S. Pat. No. 6,221,068 B1 shows a method for welding tissue in which a wound is exposed to a series of short radiation pulses, the tissue being melted in the region of the wound.

DE 689 18 155 T2 shows a surgical adhesive material, which includes the enzyme thrombin, in addition to plasma of the patient and collagen, such that the adhesive material is polymerized in an enzymatic process.

A method for producing biologically compatible, three-dimensional objects is known from EP 2 357 186 A1, in which polymerizable residues are polymerized by a two-photon or multi-photon polymerization. The polymerizable residue is intended to be biocompatible, biodegradable or bioresorbable and may be formed, for example, by a collagen component. The object to be produced may, for example, be a three-dimensional spatial element which functions as a carrier matrix for cells. The object to be produced may also constitute a structure for a synthetic production of a vessel or an organ, for example, a urethra or a kidney. In addition, the object to be produced may function as a bio-implant and, for example, may be used in the healing of wounds, in which it acts like a type of biologically degradable adhesive plaster.

The scientific publications of Ovsianikov, A.; Deiwick, A,; Van Vlierberghe, S.; Pflaum, M.; Wilhelmi, M.; Dubruel, P. and Chichkov, B.: “Laser Fabrication of 3D Gelatin Scaffolds for the Generation of Bioartificial Tissues” in Materials 2011, 4, pages 288-299 and Ovsianikov, A.; Chichkov, B. et al.: “Laser Fabrication of Three-Dimensional CAD Scaffolds from Photosensitive Gelatin for Applications in Tissue Engineering” in Biomacromolecules 2011, 12, pages 851-858 show applications for the two-photon polymerization of modified gelatins using lasers with the aid of a polymerization starter.

The article by Oujja, M.; Chichkov, B. et al.: “Three-Dimensional Microstructuring of Biopolymers by Femtosecond Laser Irradiation” in Applied Physics Letters 95, 263703, 2009,discusses the structuring of gelatins and similar substances with the aid of laser radiation.

The known methods and devices from the prior art described have various disadvantages in terms of their use for direct or indirect therapy. The known therapeutic methods using laser radiation result in the melting and denaturing of the tissue and, in some cases, the solder. Other methods require the presence of an enzyme or a comparable reaction-triggering starter substance, which limits the range of application.

The object of the present invention consists in overcoming the aforementioned disadvantages.

### SUMMARY OF THE INVENTION

The aforementioned object is achieved by a device for closing a bleeding wound of an animal or human body according to the appended claim 1. The object is further achieved by a device for processing tissue of a human or animal body according to the appended subordinate claim 4.

The device according to the invention for closing a bleeding wound of an animal or human body is used, in particular, to quickly and safely close an open bleeding wound, without damaging the rest of the tissue in the region of the wound. The device includes, first of all, a laser for irradiating the blood in the wound with infrared laser radiation. The laser radiation in this case may also comprise visible light, in particular, also red light. According to the invention, the laser radiation of the laser may be adjusted so that a two- or multi-photon absorption occurs in the irradiated regions of the blood, as a result of which the blood in the irradiated regions polymerizes into a solidified biopolymer and closes the wound. Consequently, the laser radiation of the laser is measured so that a two- or multi-photon absorption may be carried out in irradiated regions of the blood, by means of which the blood in the irradiated regions may be polymerized or solidified so that the wound may be closed. In this case, the laser is preferably designed so that the blood may be polymerized in a structured way to form the biopolymer. Thus, the device according to the invention is suitable for producing specific structures in the blood of the bleeding wound which, owing to their consistency and form, close the wound. This polymerization occurs enzyme-free and without a starter substance supplied in addition for triggering the polymerization. The invention is based on the finding that blood, or even blood plasma, may be polymerized by a two- or multi-photon polymerization using infrared light, which is comparable to a coagulation process. In particular, the blood or blood plasma may be specifically and locally structured through polymerization, the biopolymer formed adhering to blood cells, or other cells as well, and forming a solidified structure. In this context, the term biopolymer is understood to mean that polymerization of the blood results in a bio-based native polymer. In particular, the biopolymer is not denatured.

Furthermore, the device according to the invention may be used to connect parts of tissue, tissue flaps to one another. With the aid of the device, it is thus possible, similar to stitching techniques (for example, the matrix stitch), to connected tissue flaps to one another or to adhere parts of the tissue to one another to relieve tissue tensions, for example, to relieve mechanical tensile stresses. Combinations using known surgical instruments or stitching and clamping techniques, are also possible.

The device according to the invention also comprises an applicator device for applying a cell adhesive fluid to the open wound. The cell adhesive fluid is preferably native. Like the blood, the cell adhesive fluid has the property that a two- or multi-photon absorption takes place in the cell adhesive fluid as a result of an irradiation with infrared laser radiation, as a result of which the cell adhesive fluid polymerizes in the irradiated regions to form a solidified biopolymer.

This biopolymer is similarly not denatured. Because of the cell adhesive fluid, it is also possible to produce structures in the open wound in order to facilitate the closing of the wound.

The applicator device is preferably designed for spraying the cell adhesive fluid in one spray direction. In this way, it is possible to introduce the cell adhesive fluid evenly and selectively into the open wound.

Additional subject matter of the invention is defined by a method for closing a bleeding wound of an animal or human body, in particular a method for quickly and safely closing an open bleeding wound. In this method, the blood in the wound is irradiated with infrared laser radiation in order to trigger a two- or multi-photon absorption in the irradiated regions of the blood, as a result of which the blood in the irradiated regions polymerizes into a solidified biopolymer and closes the wound. In this method only native substances, such as blood and, if necessary, an additional native cell adhesive fluid, are used. No enzymes such as, for example, thrombin are supplied. In particular, there is no denaturing of the blood, of the tissue in the region of the wound and of the optionally present cell adhesive fluid.

Additional subject matter of the invention is defined by a method for adjusting the device according to the invention for closing a bleeding wound of an animal or human body. In this method, the laser is adjusted so that an irradiation of blood causes a two- or multi-photon absorption in the irradiated regions of the blood to occur, as a result of which the blood in the irradiated regions polymerizes into a solidified biopolymer.

The device according to the invention for processing tissue of a human or animal body first of all comprises an applicator device for applying a native cell adhesive fluid to the tissue to be processed. Consequently, the applicator device is designed in particular for applying a cell adhesive fluid which is not denatured and is compatible with respect to the body to be processed. Thus, unlike gelatine and the like, the cell adhesive fluid is not denatured nor is it chemically modified. With the invention, it is possible for the first time to forgo any chemical modification of the tissue. The device further comprises a laser for irradiating the applied cell adhesive fluid with infrared laser radiation. The laser radiation of the laser may be adjusted so that a two- or multi-photon absorption takes place in irradiated regions of the applied cell adhesive fluid, as a result of which the applied cell adhesive fluid in the irradiated regions polymerizes into a solidified but not denatured polymer and forms a modification to the tissue. Consequently, the laser radiation of the laser is measured so that a two- or multi-photon absorption may be carried out in irradiated regions of the applied cell adhesive fluid, as a result of which the applied cell adhesive fluid in the irradiated regions may be polymerized with no denaturing and may be solidified so that a modification may be formed on the tissue. The modification is a consolidated structure which is preferably designed to fulfill a therapeutic purpose in the human or animal body. Polymerization occurs enzyme-free and with no additionally supplied starter substance for triggering polymerization. The invention is based inter alia on the finding that native substances such as, for example, native collagen may be polymerized with infrared light by means of a two- or multi-photon polymerization. In particular, the native substance in the form of a cell adhesive fluid may be specifically and locally structured through polymerization, the resultant biopolymer adhering to cells of the body and forming a solidified structure. In this context, the term biopolymer is understood to mean that polymerization of the native substance results in a bio-based native polymer. In particular, the biopolymer is not denatured.

The device according to the invention is preferably designed for closing internal injuries, for example, for closing an organ tear. In this case, the modification to the tissue is formed by a tissue connection, in particular by an adhesive tissue connection in order to close the internal injury. The tissue connection is formed neither in an enzymatic process nor in a denaturing process.

In another preferred embodiment, the device according to the invention is designed for attaching a retina of an eye. In this case, the modification to the tissue is defined by a tissue connection, in particular by an adhesive tissue connection under the retina. The tissue connection is formed neither in an enzymatic process nor in a denaturing process.

In a particularly preferred embodiment of the device according to the invention, the latter is designed for creating a support of the tissue, the tissue being defined by a hollow organ or by a vessel of the human or animal body. The support may, for example, be a stent for a blood vessel. This embodiment of the device comprises an endoscopic tube to be introduced into the hollow organ or into the vessel. Protruding from the end of the tube are the laser and the applicator device. The laser radiation may, in particular, exit the end of the tube via an optic fiber. A particular advantage of this embodiment is that supports, in particular stents in vivo, may be created from the biocompatible biopolymer. This embodiment preferably also comprises a drainage device for draining blood and/or lymph fluid or other bodily fluids from the region in which the support is intended to be created. The drainage device is preferably also situated at the end of the endoscopic tube.

The applicator device, the laser and, optionally, the drainage device each preferably include at least one lead for purposes of their operation, which are fed through the endoscopic tube. This lead may be a flexible tube, an electrical or optical lead or a different feed line.

The applicator device is preferably designed for spraying the cell adhesive fluid in one spraying direction. In this way, it is possible to apply the cell adhesive fluid uniformly and selectively to the tissue to be processed. In this case, the laser is preferably oriented in the spray direction so that its laser radiation is oriented directly at the applied cell adhesive fluid.

In preferred embodiments, the applicator device is designed for spraying the cell adhesive fluid in a circular manner. As a result, it is possible to spray the cell adhesive fluid across the entire interior circumference of the vessel or the hollow organ in which the endoscopic tube is situated. In this example, the laser is preferably circularly focused in order to effect uniform polymerization also across the entire interior circumference. Preferably, the circular shape of the applicator device and the circular shape of the laser beam are aligned perpendicularly to the axis of the endoscopic tube and coaxially with this axis.

Additional subject matter of the invention is defined by a method for processing tissue of a human or animal body. In this method, a cell adhesive fluid is first applied to the tissue to be processed. The cell adhesive fluid is native and not denatured. It constitutes a precursor for a biopolymer. In a second step of the method, the applied cell adhesive fluid is irradiated with infrared laser radiation, such that a two- or multi-photon absorption occurs in irradiated regions of the applied cell adhesive fluid, as a result of which the applied cell adhesive fluid in the irradiated regions polymerizes into a solidified but non-denatured biopolymer and forms a modification to the tissue. This method is preferably carried out in the absence of enzymes, such as thrombin. Nor are any starter substances supplied which trigger polymerization.

Additional subject matter of the invention is defined by a method for adjusting the device according to the invention for processing tissue of a human or animal body.

In this method, the laser is adjusted so that an irradiation of the applied cell adhesive fluid causes a two- or multi-photon absorption to take place in the irradiated regions of the cell adhesive fluid, as a result of which the cell adhesive fluid in the irradiated regions polymerizes into a solidified biopolymer.

The following description of preferred embodiments relates both to the device according to the invention for closing a bleeding wound of an animal or human body and to the device according to the invention for processing tissue of a human or animal body.

The device according to the invention is preferably designed as a medical or veterinary instrument.

The device according to the invention is preferably not suited for supplying an enzyme, such as thrombin, required for the biological polymerization. The device according to the invention is also preferably not suited for supplying a starter substance which triggers polymerization.

The infrared laser radiation of the laser is preferably adjustable or measured in such a way that no denaturing of the blood or of the tissue and of the rest of the body occurs in the region of the wound or in the region of the tissue to be processed. One advantage of the device according to the invention is, namely, that its application does not result in the melting of the tissue or in similar denaturing processes. The infrared laser radiation is preferably adjustable so that the temperature in the region of the wound or of the region of the tissue to be processed remains less than 65° C., particularly preferably less than 55° C. In other particularly preferred embodiments of the device according to the invention, the output of the laser is limited in such a way that the temperature in the region of the wound or of the tissue to be processed remains less than 44° C.

The laser radiation of the laser preferably has a wave length in the near-infrared range IR-A of 780 nm to 1600 nm, particularly preferably up to 1400 nm. The radiation may, however, extend beyond this range, for example, into the visible red range.

The laser is preferably formed by a pulse laser. The pulses last preferably between 50 fs and 500 fs, particularly preferably (100±20) fs.

The laser preferably has an output of less than 2 W relative to a continuous operation. The output relative to a continuous operation is preferably between 10 mW and 1 W, particularly preferably between 50 mW and 200 mW.

In a preferred embodiment, the laser and laser system exhibit properties which adjust the propagation through pulse elongation in the endoscopic laser as a result of a negative sign (negative chirping) such that the desired pulse duration is set at the application site.

Particularly preferred embodiments of the device according to the invention also include a positioning device for positioning the laser relative to the wound to be closed or relative to the region of tissue to be processed. With the aid of the positioning device, it is possible to precisely effect locally the solidification to be achieved, i.e. the structuring to be achieved.

The positioning device is preferably defined by a focusing laser, with the aid of which it is possible to optically control the positioning of the laser. For this purpose, the device preferably also comprises a control unit with which the laser and the focusing laser may be alternately operated.

The applicator device preferably includes a flexible arm, at the end of which a nozzle is arranged for emitting the cell adhesive fluid. In this way, the applicator device may be conveniently aligned.

The cell adhesive fluid is preferably formed by a precursor of a biopolymer. This involves particularly preferably a native cell adhesive fluid originating from the body to be treated or is at least biocompatible with the latter.

The native cell adhesive fluid is preferably formed by native cells of the body to be treated, by native albumin, native blood cells, native fibrinogen, native blood plasma and/or native collagen. The cell adhesive fluid is also preferably formed by a solution of one of the aforementioned native substances, for example, by a solution of a native collagen.

In embodiments in which fibrinogen is used as a precursor of the biopolymer fibrin, fibrinogen polymerizes into fibrin, as is also ultimately the case in biological processes, in particular, in the case of blood coagulation. The invention is based on the finding that such polymerization may also be triggered by a two- or multi-photon excitation, for which purpose the fibrinogen must be irradiated with IR laser radiation. Fibrinogen or also Factor I involves a soluble glycoprotein having a high molecular weight of approximately 340 kDal, which occurs in the blood plasma. It consists of three non-identical pairs of polypeptide chains (Aα, Bβ γ)₂, which are bound by covalent disulfide bridges. The amino terminal regions of the six polypeptides are arranged in close spatial proximity via disulfide bridges, whereas the carboxyl ends are further dispersed. The A- and B- parts of the Aα- and Bβ-chains are the fibrino-peptides A and B which exhibit a surplus of negative charges. This facilitates the solubility of fibrinogen in plasma and, due to the electrostatic repulsion, also prevents an aggregation of fibrinogen molecules. The conversion of soluble fibrinogen to polymeric fibrin is one of the most important steps in the blood coagulation process and is normally catalyzed by thrombin. Serine protease in the form of thrombin splits the small fibrino peptides A and B (16 and 14 amino acids, respectively) from the high-molecular fibrinogen. As a result, bonding sites are exposed which allow the molecule, now referred to as fibrin, to spontaneously cluster together to form long-chained polymers. This aggregation is also promoted by the elimination of the surplus of negative charges. Subsequent linkages between the amide group of glutamines and the ε-amino group of lysines by a transglutaminase result in a cross-linking of the previously polymerized fibrin fibers to form a stable structure, called thrombus. This polymerization of the soluble fibrinogen to thrombus, stabilized via cross-linking initialized and terminated via a complex enzyme cascade, may in this embodiment of the method according to the invention occur completely non-enzymatically on the basis of the fibrinogen. This enables an enzyme-free polymerization according to the invention, in particular in the absence of thrombin, whereas the natural biological process requires the thrombin enzyme. The initial formation of the long-chained polymers occurs with the aid of two- or multi-photon polymerization in the manner described. A subsequent chemical linkage is preferably also enabled, which is also described further below in connection with the stabilization of collagen.

Native collagen, when it is used as a precursor of the biopolymer formed by polymerized collagen, polymerizes like the fibrinogen as a result of a two- or multi-photon absorption or two- or multi-photon excitation, which is caused by correspondingly measured IR laser radiation. In contrast to the natural process, no cross-linking agent is required in such case, so that a supplying of cross-linking agents is preferably avoided according to the invention.

The native collagen preferably has a triple helix structure with a peptide sequence motif -Gly-Xaa-Yaa- in one primary structure with at least a fraction of proline at the Xaa position and with at least a fraction of hydroxiproline at the Yaa position. Such collagen is suitable for polymerizing to form a biocompatible polymer.

The polymerized collagen preferably forms fibrils.

The collagen providable by the applicator device preferably also includes covalently bonded polyethylene glycol residues of the composition —O—(CH₂CH₂—O—)ₙ with 2≦n≦400, by means of which the structure of the collage is stabilized.

The collagen providable by the applicator device is preferably made to react with 2-bromoethylamine, ethyleneimine, N-(β-iodoethyl)trifluoracetamide and/or 2-aminoethyl-2′-amino-ethanethiolsulfonate, in order to modify sulfhydryl groups of the collagen, thereby stabilizing the structure of the collagen.

The collagen providable by the applicator device is preferably made to react with disuccinimidyl suberate (DSS); dithiobis[succinimidyl proprionate] (DSP); synonim 3,3′-dithiobis-(3-sulfo-N-hydroxy- succinimidylpropionate) disodium (DTSSP) and/or sulfosuccinimidyl 2-(biotinamido)-ethyl-1, 3-dithiopropionate (Sulfo-NHS-SS-biotin) providable by the or one additional applicator device, in order to modify amino residues of the collagen, thereby stabilizing the structure of the collagen.

The cell adhesive fluid in the form of a precursor may be used in various forms. The precursor may, for example, be provided as a diluted solution or also as a diluted, buffered solution in an aqueous medium. The precursor may also be provided as a diluted solution in a non-aqueous medium. The precursor, in particular the collagen, is preferably used in a concentrated form as a gel-like substance.

Additional preferred embodiments of the devices according to the invention include features which are specified as essential or as preferable for the method according to the invention. In particular, the devices according to the invention are preferably designed for carrying out steps which are specified as essential or preferable for the method according to the invention. In particular, the methods according to the invention are preferably designed for applying the devices according to the invention, including preferred embodiments.

Additional advantages, details and refinements of the invention will become apparent from the following description of preferred embodiments of the device according to the invention.

### BRIEF DESCRIPTION OF THE DRAWINGS

FIG. 1 shows a first embodiment of a device according to the invention for creating a stent;

FIG. 2 shows a preferred embodiment of the device according to the invention for creating a stent;

FIG. 3 shows a particularly preferable embodiment of the device according to the invention for creating a stent;

FIG. 4 shows a sectional view of the device shown in FIG. 3; and

FIG. 5 shows a coupling region of the device shown in FIG. 3.

### DETAILED DESCRIPTION

FIG. 1 shows a first embodiment of a device according to the invention for creating a stent. The device is designed to be introduced endoscopically into a vessel, in particular, a blood vessel of a human or an animal. For this purpose, the device includes an endoscopic tube 01, at the front end 02 of which a spray nozzle 03 of an applicator device and a laser 04 appear. The spray nozzle 03 of the applicator device is used to spray a cell adhesive fluid, to be applied to the interior wall of the vessel to be treated. The laser 04 is designed to irradiate the applied cell adhesive fluid with infrared laser radiation, in order to cause a two- or multi-photon absorption in the cell adhesive fluid. In the embodiment shown, the laser 04 and the spray nozzle 03 are arranged in parallel. The laser beam of the laser 04 may, for example, be radially or linearly focused.

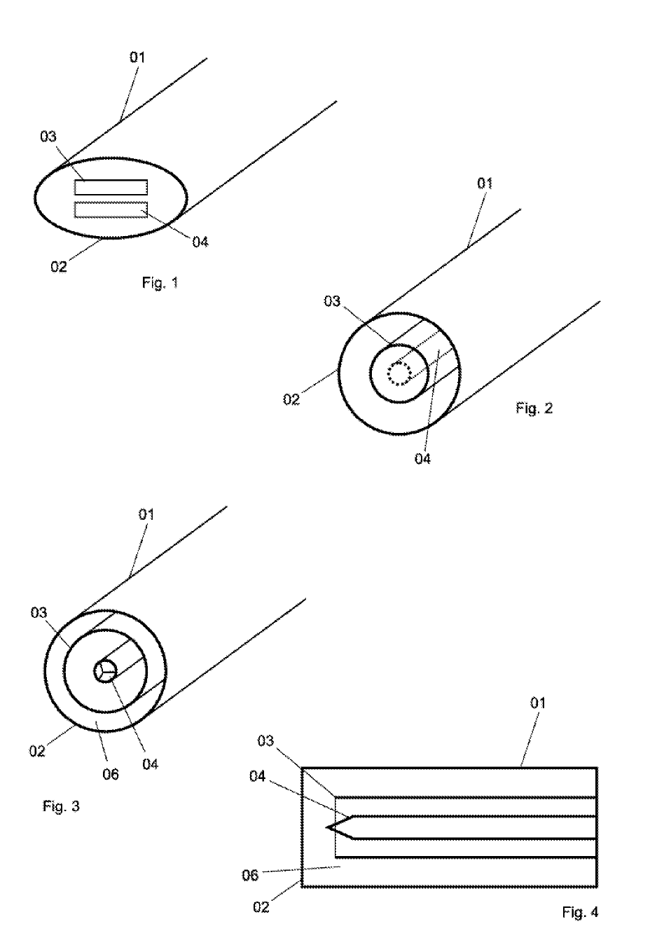

FIG. 2 shows a preferred embodiment of the device according to the invention for creating a stent. This embodiment has the same scope of application as the embodiment shown in FIG. 1. This embodiment in turn also includes the endoscopic tube 01, at the front end 02 of which the spray nozzle 03 of an applicator device and the laser 04 appear. In this embodiment, the laser 04 is situated behind the spray nozzle 03 such that the laser beam of the laser 04 radiates through the cell adhesive fluid to be sprayed.

FIG. 3 shows a particularly preferred embodiment of the device according to the invention for creating a stent, which has the same scope of application as the embodiment shown in FIG. 1. This embodiment also includes the endoscopic tube 01, at the front end 02 of which the spray nozzle 03 of an applicator device and the laser 04 emerge. In this embodiment, the spray nozzle 03 is circular in design and arranged coaxially relative to the laser 04. The circular spray nozzle 03 is designed to spray the cell adhesive fluid in a circular pattern. The laser 04 is circularly focused so that the laser beam of the laser 04 uniformly strikes the circularly sprayed cell adhesive fluid. Also situated at the front end 02 of the endoscopic tube 01 is drainage opening 06, through which blood and other bodily fluids may be suctioned from the region of the stent to be created.

FIG. 4 shows a cross-sectional view of the device shown in FIG. 3.

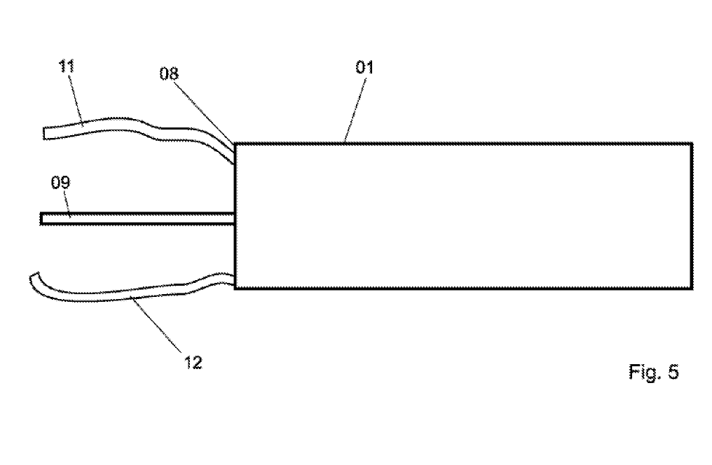

FIG. 5 shows a coupling region of the device shown in FIG. 3. The coupling region is formed at the rear end 08 of the endoscopic tube 01, which is situated opposite the front end 02 shown in FIG. 3. Emerging at the rear end 08 of the endoscopic tube 01 is an optical fiber 09 for the laser 04 (shown in FIG. 3), a feed line 11 for the applicator device and a drainage line 12. The feed line 11 is used to feed the cell adhesive fluid in such a way that it is able pass through the endoscopic tube 01, exiting at the spray nozzle 03 (shown in FIG. 3). The drainage line 12 is used to drain the bodily fluid discharged via the drainage opening 06 (shown in FIG. 3) through the endoscopic tube 01.

### LIST OF REFERENCE NUMERALS

01—endoscopic tube
02—front end
03—spray nozzle
04—laser
05 - - -
06—drainage opening
107 - - -
08—rear end
09—optical fiber
10 - - -
11—feed line
12—drainage line

1. A device for closing a bleeding wound of an animal or human body, comprising:
a laser for irradiating the blood in the wound with infrared laser radiation,
wherein the infrared laser radiation of the laser may be adjusted so that a two- or multi-photon absorption takes place in the irradiated regions of the blood, as a result of which the blood in the irradiated regions polymerizes into a solidified biopolymer and closes the wound.

2. The device according to claim 1, further comprising an applicator device for applying a cell adhesive fluid in the open wound.

3. The device according to claim 1, wherein the infrared laser radiation of the laser may be adjusted so that a two- or multi-photon absorption takes place in the irradiated regions of the blood, as a result of which the blood in the irradiated regions polymerizes with no denaturing to form a solidified biopolymer and closes the wound.

4. A device for processing tissue of a human or animal body, comprising:
an applicator device for applying a cell adhesive fluid to the tissue to be processed; and
a laser for irradiating the applied cell adhesive fluid with infrared laser radiation, wherein the infrared laser radiation of the laser may be adjusted so that a two- or multi-photon absorption takes place in the irradiated regions of the applied cell adhesive fluid, as a result of which the applied cell adhesive fluid in the irradiated regions polymerizes into a solidified biopolymer and forms a modification to the tissue.

5. The device according to claim 4, wherein the device is designed for creating a support constituting the modification to the vessel constituting the tissue, and further comprises an endoscopic tube to be introduced into the vessel, and wherein the laser and the applicator device emerge at the end of the tube.

6. The device according to claim 4, wherein the infrared laser radiation of the laser may be adjusted so that a two- or multi-photon absorption takes place in the irradiated regions of the applied cell adhesive fluid, as a result of which the applied cell adhesive fluid in the irradiated regions polymerizes with no denaturing to a solidified biopolymer and forms a modification to the tissue.

7. The device according to claim 1, wherein the device is designed as a medical instrument or as a veterinary instrument.

8. The device according to claim 1, wherein the infrared laser radiation of the laser may be adjusted so that the temperature in the region of the wound or of the region of the tissue to be processed remains less than 55° C.

9. The device according to claim 1, wherein the output of the laser is limited in such a way that the temperature in the region of the wound or of the tissue to be processed remains less than 44° C.

10. The device according to claim 1, wherein the laser radiation of the laser has a wave length in the near infrared range.

11. The device according to claim 1, wherein the laser is formed by a laser pulse, the pulses of which last between 50 fs and 200 fs.

12. The device according to claim 1, wherein the laser radiation of the laser has an output relative to a continuous operation of between 50 mW and 200 mW.

13. The device according to claim 1, wherein the device further comprises an applicator device for applying a cell adhesive fluid using a spray nozzle.

14. The device according to claim 4, wherein the device is designed as a medical instrument or as a veterinary instrument.

15. The device according to claim 4, wherein the infrared laser radiation of the laser may be adjusted so that the temperature in the region of the wound or of the region of the tissue to be processed remains less than 55° C.

16. The device according to claim 4, wherein the output of the laser is limited in such a way that the temperature in the region of the wound or of the tissue to be processed remains less than 44° C.

17. The device according to claim 4, wherein the laser radiation of the laser has a wave length in the near infrared range.

18. The device according to claim 4, wherein the laser is formed by a laser pulse, the pulses of which last between 50 fs and 200 fs.

19. The device according to claim 4, wherein the laser radiation of the laser has an output relative to a continuous operation of between 50 mW and 200 mW.

20. The device according to claim 4, wherein the device comprises an applicator device for applying a cell adhesive fluid using a spray nozzle.
